(19) United States
(12) Patent Application Publication
(10) Pub. No.: US 2020/0028088 A1
(43) Pub. Date: Jan. 23, 2020

(54) ANTHRACENE DERIVATIVES CONTAINING BENZIMIDAZOLE OR BORATE AND ORGANOELECTROLUMINESCENT DEVICE INCLUDING THE SAME

(71) Applicants: YUAN ZE UNIVERSITY, Chung-Li (TW); NICHEM FINE TECHNOLOGY CO., LTD., Jhubei City (TW); TETRAHEDRON TECHNOLOGY CORPORATION, Zhunan Township (TW); WAN HSIANG PRECISION MACHINERY CO., LTD., Kaohsiung City (TW)

(72) Inventors: Tien-Lung CHIU, Chung-Li (TW); Jiun-Haw LEE, Chung-Li (TW); Man-Kit LEUNG, Chung-Li (TW); Chi-Feng LIN, Chung-Li (TW); Liang-Ju HOU, Chung-Li (TW); Bo-An FAN, Chung-Li (TW); Chiou-Ling CHANG, Chung-Li (TW)

(21) Appl. No.: 16/194,974

(22) Filed: Nov. 19, 2018

(30) Foreign Application Priority Data
Jul. 18, 2018 (TW) 107124863

(51) Int. Cl. H01L 51/00; C07D 235/20; C09K 11/06; C07C 233/80; C07D 235/18

(52) U.S. Cl. CPC H01L 51/0052; C07D 235/20; C09K 11/06; H01L 51/5012; C07C 233/80; H01L 51/006; C07D 235/18; H01L 51/0072

(57) ABSTRACT

An organic light-emitting diode comprises a first electrode layer, a second electrode layer, and an organic luminescent unit disposed between the first electrode layer and the second electrode layer. The organic luminescent unit has an organic electroluminescent material containing anthracene group as shown in General Formula (1):

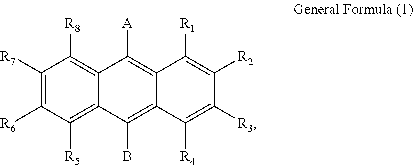

wherein A is selected from the group consisting of General Formula (2), General Formula (3) and General Formula (4):

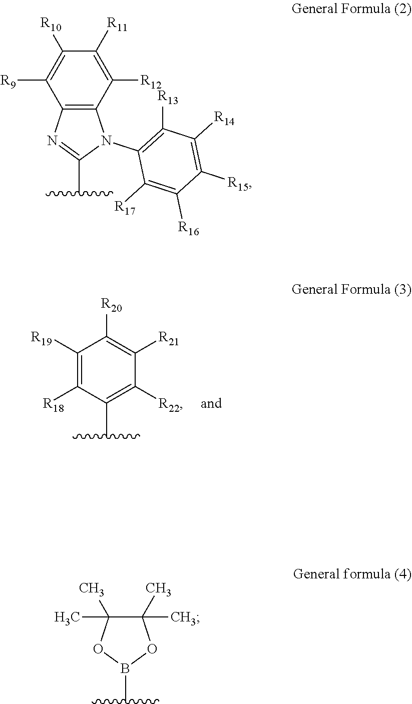

wherein B is selected from the group consisting of General Formula (5), General Formula (6) and General Formula (7):

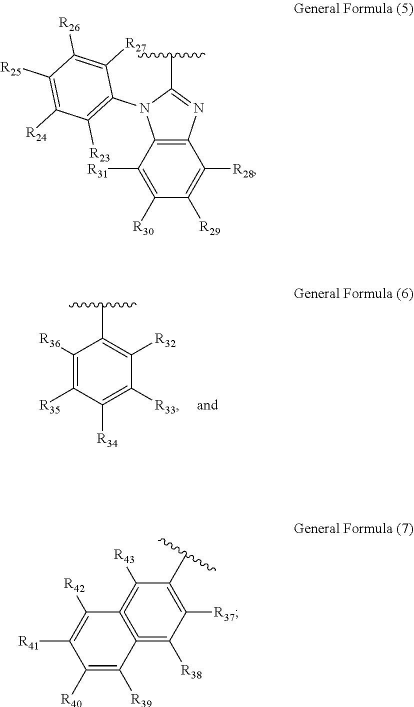

B is General Formula (5) when A is selected from the group consisting of General Formula (2) and General Formula (3);
B is selected from the group consisting of General Formula (6) and General
Formula (7) when A is General Formula (4); and wherein R₁ to R₄₃ are independently selected from the group consisting of hydrogen atom, fluorine atom, cyano group, alkyl group, cycloalkyl group, alkoxy group, haloalkyl group, thioalkyl group, silyl group and alkenyl group.

### CROSS REFERENCE TO RELATED APPLICATIONS

This Non-provisional application claims priority under 35 U.S.C. § 119(a) on Patent Application No(s). 107124863 filed in Taiwan, Republic of China on Jul. 18, 2018, the entire contents of which are hereby incorporated by reference.

### BACKGROUND

### Technology Field

The present disclosure relates to electroluminescent materials and light-emitting elements by using the same and, in particular, to electroluminescent materials containing anthracene group and organic light-emitting diodes by using the same.

### Description of Related Art

With the advances in electronic technology, a light weight and high efficiency flat display device has been developed. An organic electroluminescent device possibly becomes the mainstream of the next generation flat panel display device due to its advantages of self-luminosity, no restriction on viewing angle, power conservation, simple manufacturing process, low cost, high response speed, full color and so on.

In general, the organic electroluminescent device includes an anode, an organic luminescent layer and a cathode. When applying a direct current to the organic electroluminescent device, electron holes and electrons are injected into the organic luminescent layer from the anode and the cathode, respectively. Charge carriers move and then recombine in the organic luminescent layer because of the potential difference caused by an applied electric field. The excitons generated by the recombination of the electrons and the electron holes may excite the luminescent molecules in the organic luminescent layer. The excited luminescent molecules then release the energy in the form of light.

Nowadays, the organic electroluminescent device usually adopts a host-guest emitter system. The organic luminescent layer disposed therein includes a host material and a guest material. The electron holes and the electrons are mainly transmitted to the host material to perform recombination and thereby generate energy, and then the energy is transferred to the guest material to generate light. The guest material can be categorized into fluorescent material and phosphorescent material.

Theoretically, the internal quantum efficiency can approach 100% by using appropriate phosphorescent materials. However, the multiple atom centers of guest materials are mostly precious metals, which are difficult to synthesize and expensive, and the triplet excitons have longer lifetimes than singlet excitons, and easily have quenching therebetween when high concentration triplet excitons are generated at high current densities. The quenching of the triplet states causes a sudden drop in luminous efficiency. In addition, since the decay period of phosphorescence is long, the image may easily have ghost images. If applied to a high dynamic display screen, it will be a great defect when utilizing the phosphorescent organic light-emitting material. In addition, the electroluminescence of the phosphorescent material can only produce 25% of singlet excitons without using a mechanism to increase the fluorescence quantum yield.

There are currently two mechanisms for converting 75% of triplet excitons back to singlet states to increase their fluorescence quantum yield. One mechanism is thermally activated delayed fluorescence (TADF), and the other mechanism is triplet-triplet annihilation photon upconversion (TTA-UC).

In the current organic light-emitting diode materials by using TTA-UC as the fluorescent mechanism, the major materials include 9,10-diphenylanthracene (DPA) and 9,10-di(naphthalen-2-yl)anthracene (ADN).

Besides, the selection of organic electroluminescent material is not only based on the matching energy level but also the high temperature of decomposition to avoid pyrolysis caused by high temperature and also avoid the resulted decreasing of stability.

Accordingly, the present disclosure provides electroluminescent materials containing anthracene group and organic light-emitting diodes by using the same which have good fluorescence quantum performance and thermal stability.

### SUMMARY

In view of the foregoing, an objective of the present disclosure is to provide electroluminescent materials containing anthracene group and organic light-emitting diodes by using the same which have good fluorescence quantum performance and thermal stability.

To achieve the above objective, the present disclosure provides an organic light-emitting diode, comprising a first electrode layer, a second electrode layer, and an organic luminescent unit disposed between the first electrode layer and the second electrode layer. The organic luminescent unit has an organic electroluminescent material containing anthracene group as shown in General Formula (1):

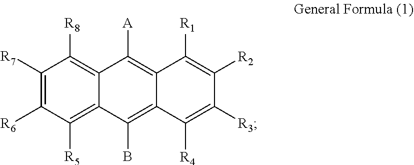

Wherein A is selected from the group consisting of General Formula (2), General Formula (3) and General Formula (4):

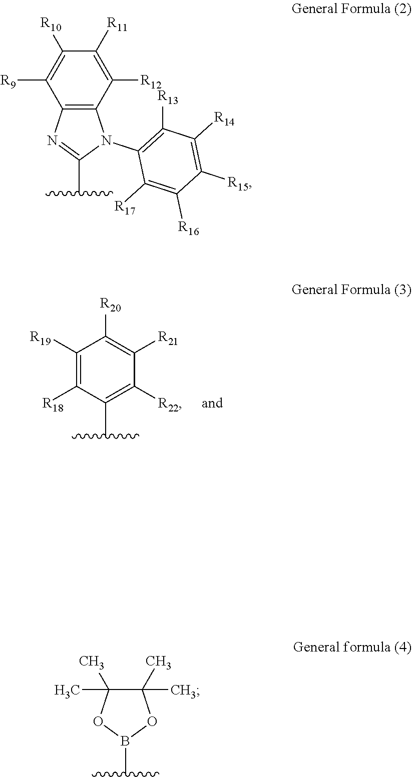

Wherein B is selected from the group consisting of General Formula (5), General Formula (6) and General Formula (7):

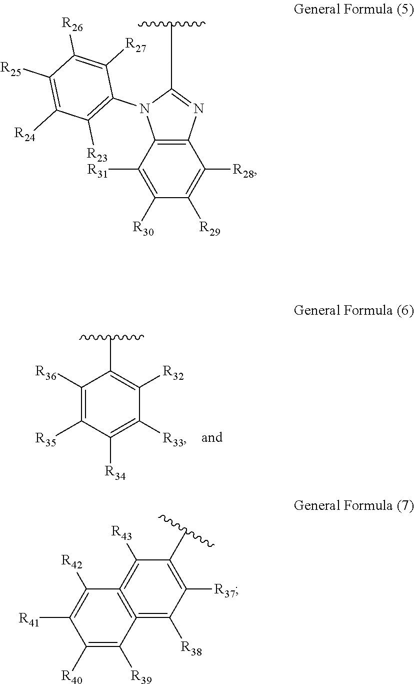

Wherein B is General Formula (5) when A is selected from the group consisting of General Formula (2) and General Formula (3);

Wherein B is selected from the group consisting of General Formula (6) and General Formula (7) when A is General Formula (4); and

Wherein R₁ to R₄₃ are independently selected from the group consisting of hydrogen atom, fluorine atom, cyano group, alkyl group, cycloalkyl group, alkoxy group, haloalkyl group, thioalkyl group, silyl group and alkenyl group.

In one embodiment, the alkyl group is selected from the group consisting of a substituted or unsubstituted straight-chain C1˜C6 alkyl group, and a substituted or unsubstituted branched-chain C3˜C6 alkyl group, the cycloalkyl group is a substituted or unsubstituted C3˜C6 cycloalkyl group, the alkoxy group is selected from the group consisting of a substituted or unsubstituted straight-chain C1˜C6 alkoxy group, and a substituted or unsubstituted branched-chain C3˜C6 alkoxy group, the haloalkyl group is selected from the group consisting of a substituted or unsubstituted straight-chain C1˜C6 haloalkyl group, and a substituted or unsubstituted branched-chain C3˜C6 haloalkyl group, the thioalkyl group is selected from the group consisting of a substituted or unsubstituted straight-chain C1˜C6 thioalkyl group, and a substituted or unsubstituted branched-chain C3˜C6 thioalkyl group, the silyl group is selected from the group consisting of a substituted or unsubstituted straight-chain C1˜C6 silyl group, and a substituted or unsubstituted branched-chain C3˜C6 silyl group, and the alkenyl group is selected from the group consisting of a substituted or unsubstituted straight-chain C2˜C6 alkenyl group, and a substituted or unsubstituted branched-chain C3˜C6 alkenyl group.

In one embodiment, the organic electroluminescent material containing anthracene group comprises a structure of any of the following Chemical Formulas (1) to (4):

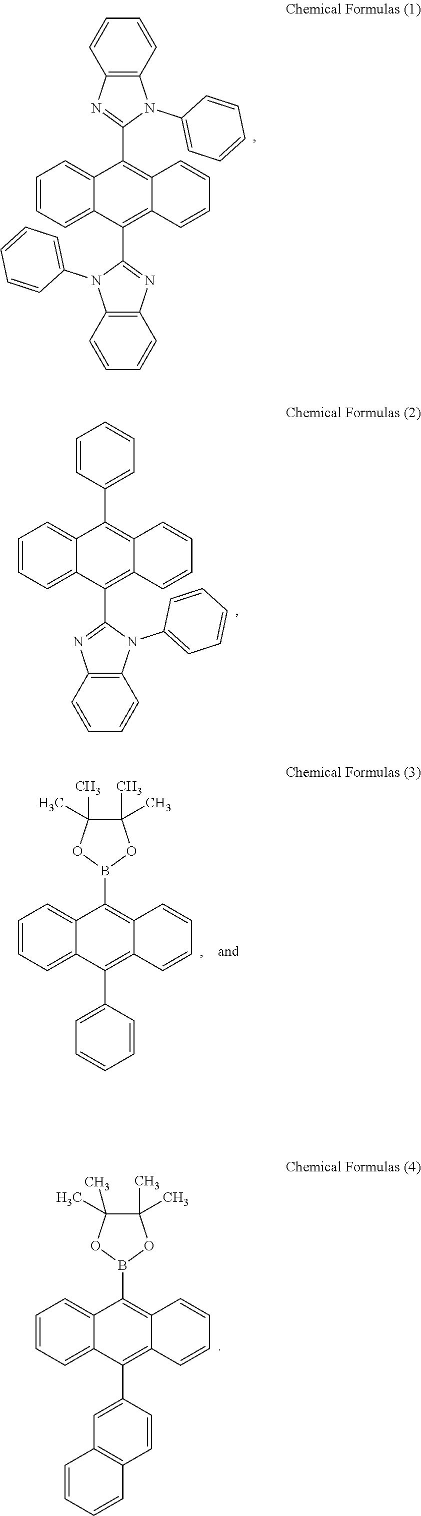 [SMILES: c1ccc(-n2c(-c3c4ccccc4c(-c4nc5ccccc5n4-c4ccccc4)c4ccccc34)nc3ccccc32)cc1 ; c1ccc(-c2c3ccccc3c(-c3nc4ccccc4n3-c3ccccc3)c3ccccc23)cc1 ; CC1(C)OB(c2c3ccccc3c(-c3ccccc3)c3ccccc23)OC1(C)C ; CC1(C)OB(c2c3ccccc3c(-c3ccc4ccccc4c3)c3ccccc23)OC1(C)C]

In one embodiment, the organic luminescent unit comprises an organic luminescent layer.

In one embodiment, the organic luminescent unit further comprises a hole transport layer and an electron transport layer, and the organic luminescent layer is disposed between the hole transport layer and the electron transport layer.

In one embodiment, the organic luminescent unit further comprises a hole injection layer, a hole transport layer, an electron transport layer and an electron injection layer, and the hole transport layer, the organic luminescent layer and the electron transport layer are sequentially disposed between the hole injection layer and the electron injection layer.

In one embodiment, the organic electroluminescent material containing anthracene group is a fluorescent organic electroluminescent material.

In one embodiment, the organic luminescent layer comprises polyvinylcarbazole and the organic electroluminescent material containing anthracene group.

In one embodiment, a doping concentration of the organic electroluminescent material containing anthracene group ranges from 20% to 40%.

To achieve the above objective, the present disclosure also provides an organic electroluminescent material containing anthracene group, comprising a structure of the following General Formula (1):

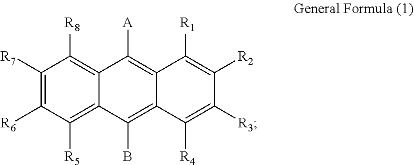

Wherein A is selected from the group consisting of General Formula (2) and General Formula (3):

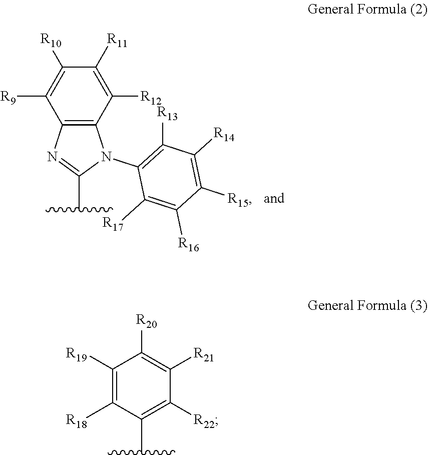

Wherein B comprises a structure of General Formula (5):

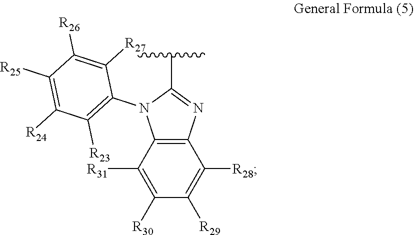

and

Wherein R₁ to R₃₁ are independently selected from the group consisting of hydrogen atom, fluorine atom, cyano group, alkyl group, cycloalkyl group, alkoxy group, haloalkyl group, thioalkyl group, silyl group and alkenyl group.

In one embodiment, the alkyl group is selected from the group consisting of a substituted or unsubstituted straight-chain C1˜C6 alkyl group, and a substituted or unsubstituted branched-chain C3˜C6 alkyl group, the cycloalkyl group is a substituted or unsubstituted C3˜C6 cycloalkyl group, the alkoxy group is selected from the group consisting of a substituted or unsubstituted straight-chain C1˜C6 alkoxy group, and a substituted or unsubstituted branched-chain C3˜C6 alkoxy group, the haloalkyl group is selected from the group consisting of a substituted or unsubstituted straight-chain C1˜C6 haloalkyl group, and a substituted or unsubstituted branched-chain C3˜C6 haloalkyl group, the thioalkyl group is selected from the group consisting of a substituted or unsubstituted straight-chain C1˜C6 thioalkyl group, and a substituted or unsubstituted branched-chain C3˜C6 thioalkyl group, the silyl group is selected from the group consisting of a substituted or unsubstituted straight-chain C1˜C6 silyl group, and a substituted or unsubstituted branched-chain C3˜C6 silyl group, and the alkenyl group is selected from the group consisting of a substituted or unsubstituted straight-chain C2˜C6 alkenyl group, and a substituted or unsubstituted branched-chain C3˜C6 alkenyl group.

In one embodiment, the organic electroluminescent material containing anthracene group comprises a structure of any of the following Chemical Formulas (1) to (2):

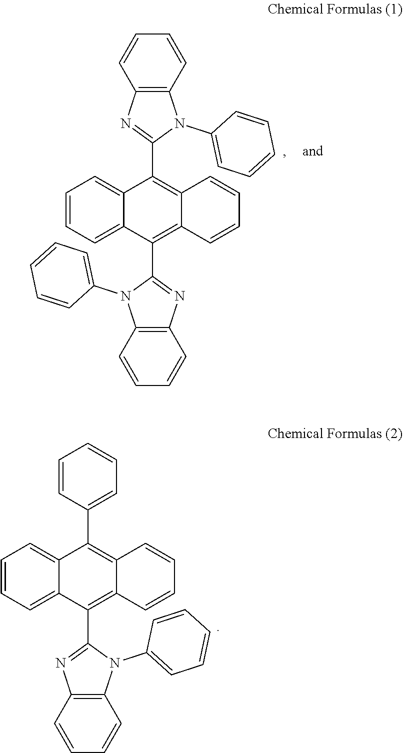 [SMILES: c1ccc(-n2c(-c3c4ccccc4c(-c4nc5ccccc5n4-c4ccccc4)c4ccccc34)nc3ccccc32)cc1 ; c1ccc(-c2c3ccccc3c(-c3nc4ccccc4n3-c3ccccc3)c3ccccc23)cc1]

As mentioned above, in the electroluminescent materials containing anthracene group and organic light-emitting diodes by using the same according to the present disclosure, it utilizes anthracene as a core structure. Benzimidazole having electron transport function is introduced to synthesize the electroluminescent materials containing anthracene group, which have good fluorescence quantum performance and thermal stability. Thus, the electroluminescent materials containing anthracene group are suitable for manufacturing the organic light-emitting diodes with good fluorescence quantum performance and thermal stability.

### DESCRIPTION OF THE DRAWINGS

The disclosure will become more fully understood from the detailed description and accompanying drawings, which are given for illustration only, and thus are not limitative of the present disclosure, and wherein:

FIG. 1 is a sectional view of an organic light-emitting diode according to a first embodiment of this disclosure;

FIG. 2 is a sectional view of an organic light-emitting diode according to a second embodiment of this disclosure; and

FIG. 3 is a sectional view of an organic light-emitting diode according to a third embodiment of this disclosure.

### DETAILED DESCRIPTION OF THE DISCLOSURE

The present disclosure will be apparent from the following detailed description, which proceeds with reference to the accompanying drawings, wherein the same references relate to the same elements.

Organic Light-Emitting Diodes

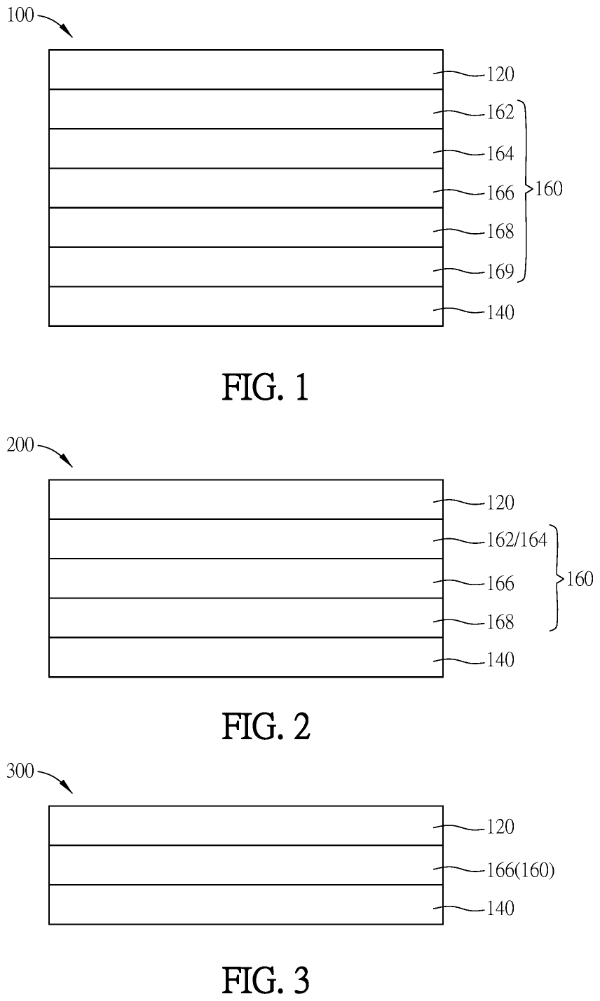

Please refer to FIG. 1, an organic light-emitting diode 100 according to the second embodiment of the disclosure includes a first electrode layer 120, a second electrode layer 140 and an organic luminescent unit 160. In the embodiment, the first electrode layer 120 can be a transparent electrode material, such as indium tin oxide (ITO), and the second electrode layer 140 can be a metal, transparent conductive substance or any other suitable conductive material. On the other hand, the first electrode layer 120 can also be a metal, transparent conductive substance or any other suitable conductive material, and the second electrode layer 140 can also be a transparent electrode material. Overall, at least one of the first electrode layer 120 and the second electrode layer 140 of the embodiment is a transparent electrode material, so that the light emitted from the organic luminescent unit 160 may pass through the transparent electrode, thereby enabling the organic light-emitting diode 100 to emit light.

In addition, please also refer to FIG. 1, the organic luminescent unit 160 can comprise a hole injection layer 162, a hole transport layer 164, an organic luminescent layer 166, an electron transport layer 168 and an electron injection layer 169. The hole transport layer 164, the organic luminescent layer 166 and the electron transport layer 168 are sequentially disposed between the hole injection layer 162 and the electron injection layer 169.

Herein, the materials of the hole injection layer 162 may be poly(3,4-ethylenedioxythiophene) polystyrene sulfonate (PEDOT:PSS) or PEDOT. The thickness of the hole transport layer 162 of the embodiment is, for example, less than or equal to 40 nm.

The materials of the hole transport layer 164 may be 1,1-Bis[4-[N,N′-di(p-tolyl)amino]phenyl]cyclohexane (TAPC), N,N-bis-(1-naphthyl)-N,N-diphenyl-1,1-biphenyl-4,4-diamine (NPB), or N—N′-diphenyl-N—N′bis(3-methylphenyl)-[1-1′-biphenyl]-4-4′-diamine (TPD). In the embodiment, the thickness of the hole transport layer 164 ranges, for example, from 0 nm to 100 nm. The hole injection layer 162 and the hole transport layer 164 may further increase the injection rate of the electron hole from the first electrode layer 120 to the organic luminescent layer 166, and reduce the driving voltage of the organic light-emitting diode 100.

In addition, the thickness of the organic luminescent layer 166 of the embodiment is, for example, between 5 nm and 60 nm. For example, the thickness of the organic luminescent layer 166 of the embodiment is 30 nm. The organic luminescent layer 166 includes an organic electroluminescent material containing anthracene group and polyvinylcarbazole. The organic electroluminescent material containing anthracene group has a structure of the following General Formula (1):

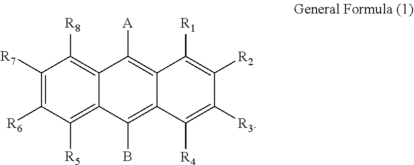

Wherein A is selected from the group consisting of General Formula (2), General Formula (3) and General Formula (4):

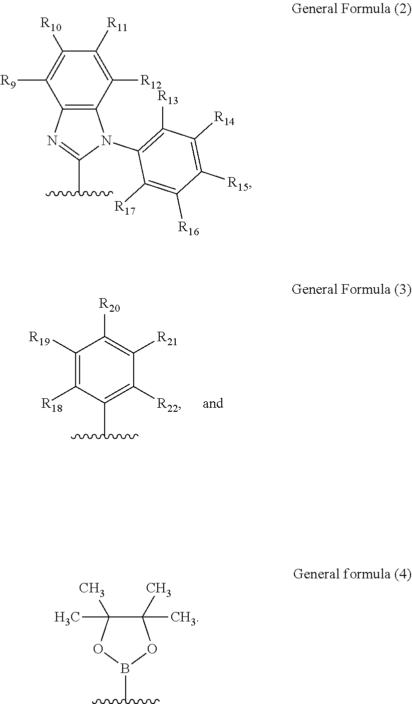

Wherein B is selected from the group consisting of General Formula (5), General Formula (6) and General Formula (7):

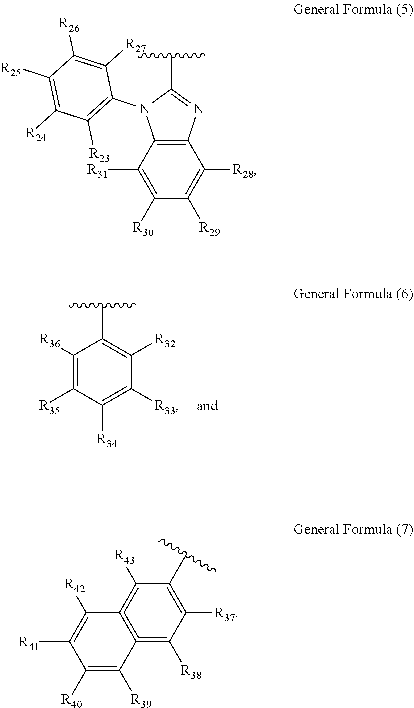

Wherein B is General Formula (5) when A is selected from the group consisting of General Formula (2) and General Formula (3).

Wherein B is selected from the group consisting of General Formula (6) and General Formula (7) when A is General Formula (4).

Wherein R₁ to R₄₃ are independently selected from the group consisting of hydrogen atom, fluorine atom, cyano group, alkyl group, cycloalkyl group, alkoxy group, haloalkyl group, thioalkyl group, silyl group and alkenyl group.

In this embodiment, the alkyl group is selected from the group consisting of a substituted or unsubstituted straight-chain C1˜C6 alkyl group, and a substituted or unsubstituted branched-chain C3˜C6 alkyl group, the cycloalkyl group is a substituted or unsubstituted C3˜C6 cycloalkyl group, the alkoxy group is selected from the group consisting of a substituted or unsubstituted straight-chain C1˜C6 alkoxy group, and a substituted or unsubstituted branched-chain C3˜C6 alkoxy group, the haloalkyl group is selected from the group consisting of a substituted or unsubstituted straight-chain C1˜C6 haloalkyl group, and a substituted or unsubstituted branched-chain C3˜C6 haloalkyl group, the thioalkyl group is selected from the group consisting of a substituted or unsubstituted straight-chain C1˜C6 thioalkyl group, and a substituted or unsubstituted branched-chain C3˜C6 thioalkyl group, the silyl group is selected from the group consisting of a substituted or unsubstituted straight-chain C1˜C6 silyl group, and a substituted or unsubstituted branched-chain C3˜C6 silyl group, and the alkenyl group is selected from the group consisting of a substituted or unsubstituted straight-chain C2˜C6 alkenyl group, and a substituted or unsubstituted branched-chain C3˜C6 alkenyl group.

A preferred example is the compound of Chemical Formula (1), DiBizAn, where A is the General Formula (2), B is the General Formula (5), R₁ to R₁₇ and R₂₃ to R₃₁ are independent hydrogen atoms.

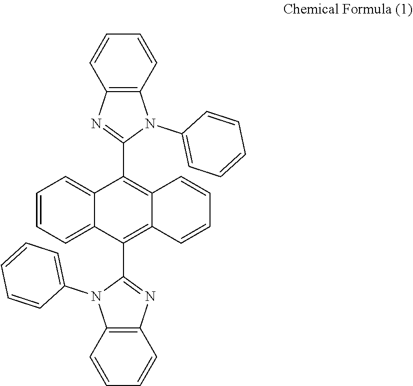

Alternatively, another preferred example is the compound of Chemical Formula (2), monoBizAn, where A is the General Formula (3), B is the General Formula (5), R₁ to R₈ and R₁₈ to R₃₁ are independent hydrogen atoms.

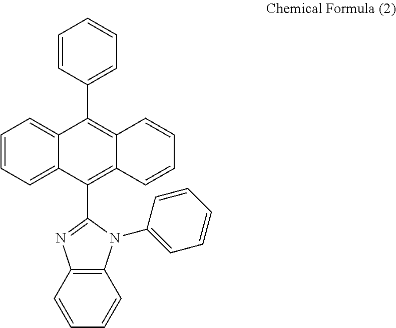

Alternatively, another preferred example is the compound of Chemical Formula (3), PhBorAn, where A is the General Formula (4), B is the General Formula (6), R₁ to R₈ and R₃₂ to R₃₆ are independent hydrogen atoms.

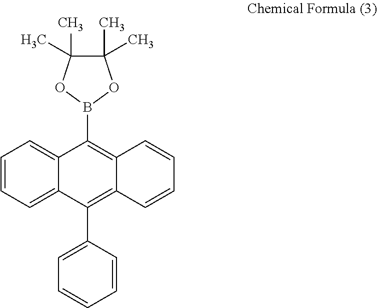

Alternatively, another preferred example is the compound of Chemical Formula (4), NpBorAn, where A is the General Formula (4), B is the General Formula (7), R₁ to R₈ and R₃₇ to R₄₃ are independent hydrogen atoms.

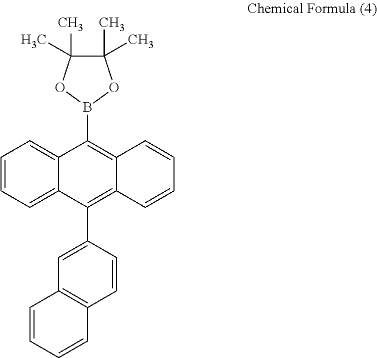

In Chemical Formulas (1) to (4), anthracene is utilized as a core group, and benzimidazole having electron transport function is introduced to synthesize the electroluminescent materials containing anthracene group, which have good fluorescence quantum performance and thermal stability. Thus, the electroluminescent materials containing anthracene group are suitable for manufacturing the organic light-emitting diodes with good fluorescence quantum performance and thermal stability.

In this embodiment, the doping concentration of the electroluminescent materials containing anthracene group can range from 20% to 40%. For example, the doping concentration of the electroluminescent materials containing anthracene group can be 20%, 30% or 40%.

In addition, the material of the electron transport layer 168 may be, for example but not limited to, a metal complex, such as Tris-(8-hydroxy-quinoline)aluminum (Alq₃), bis(10-hydroxybenzo-[h]quinolinato)beryllium (BeBq₂) and so on, or a heterocyclic compound, such as 2-(4-Biphenylyl)-5-(4-tert-butylphenyl)-1,3,4-oxadiazole (PBD), 3-(4-Biphenyl)-4-phenyl-5-tert-butylphenyl-1,2,4-triazole (TAZ), 2,2′,2″-(1,3,5-Benzinetriyl)-tris(1-phenyl-1-H-benzimidazole) (TPBI), diphenylbis(4-(pyridin-3-yl)phenyl)silane (DPPS), Bathophenanthroline (Bphen) and so on. In the embodiment, the thickness of the electron transport layer 168 may be, for example, less than 100 nm. The electron transport layer 168 can facilitate the transfer of electrons from the second electrode layer 140 to the organic luminescent layer 166 to increase the transport rate of electrons. Moreover, the material of the electron injection layer 169 may be, for example, LiF. The thickness of the electron injection layer 169 may be, for example, 1 nm.

In addition, FIG. 2 is a sectional view of an organic light-emitting diode 200 according to the second embodiment of the disclosure. The configuration of the organic light-emitting diode 200 is substantially similar with that of the organic light-emitting diode 100, and same elements have substantial the same characteristics and functions. Therefore, the similar references relate to the similar elements, and detailed explanation is omitted hereinafter.

Please refer to FIG. 2, in the embodiment, the organic luminescent unit 160 can comprise a hole injection layer 162 or a hole transport layer 164, an organic luminescent layer 166 and an electron transport layer 168. The organic luminescent layer 166 is disposed between the electron transport layer 168 and the hole injection layer 162 or hole transport layer 164.

In addition, FIG. 3 is a sectional view of an organic light-emitting diode 300 according to the third embodiment of the disclosure. The configuration of the organic light-emitting diode 300 is substantially similar with that of the organic light-emitting diode 100, and same elements have substantial the same characteristics and functions. Therefore, the similar references relate to the similar elements, and detailed explanation is omitted hereinafter.

Please refer to FIG. 3, in the embodiment, the organic luminescent unit 160 can comprise an organic luminescent layer 166.

The configuration of the organic light-emitting diode according to the disclosure is not limited to what is disclosed in the first, second or third embodiment. The first, second and third embodiments are for illustrations only.

Electroluminescent Materials Containing Anthracene Group

A fourth embodiment of the present disclosure provides an organic electroluminescent material containing anthracene group, comprising a structure of the following General Formula (1):

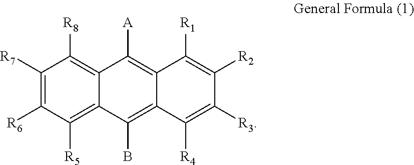

Wherein A is selected from the group consisting of General Formula (2) and General Formula (3):

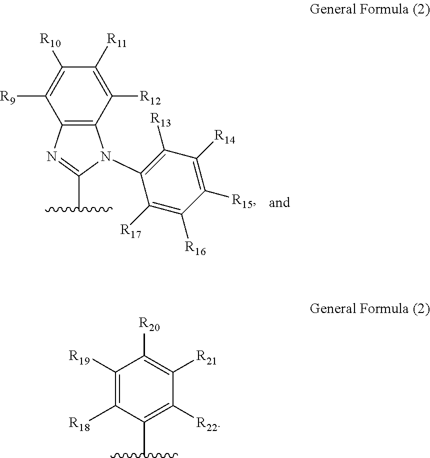

Wherein B comprises a structure of General Formula (5):

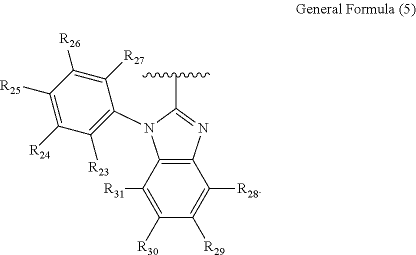

Wherein R₁ to R₃₁ are independently selected from the group consisting of hydrogen atom, fluorine atom, cyano group, alkyl group, cycloalkyl group, alkoxy group, haloalkyl group, thioalkyl group, silyl group and alkenyl group.

In this embodiment, the alkyl group is selected from the group consisting of a substituted or unsubstituted straight-chain C1˜C6 alkyl group, and a substituted or unsubstituted branched-chain C3˜C6 alkyl group, the cycloalkyl group is a substituted or unsubstituted C3˜C6 cycloalkyl group, the alkoxy group is selected from the group consisting of a substituted or unsubstituted straight-chain C1˜C6 alkoxy group, and a substituted or unsubstituted branched-chain C3˜C6 alkoxy group, the haloalkyl group is selected from the group consisting of a substituted or unsubstituted straight-chain C1˜C6 haloalkyl group, and a substituted or unsubstituted branched-chain C3˜C6 haloalkyl group, the thioalkyl group is selected from the group consisting of a substituted or unsubstituted straight-chain C1˜C6 thioalkyl group, and a substituted or unsubstituted branched-chain C3˜C6 thioalkyl group, the silyl group is selected from the group consisting of a substituted or unsubstituted straight-chain C1˜C6 silyl group, and a substituted or unsubstituted branched-chain C3˜C6 silyl group, and the alkenyl group is selected from the group consisting of a substituted or unsubstituted straight-chain C2˜C6 alkenyl group, and a substituted or unsubstituted branched-chain C3˜C6 alkenyl group.

The structure of General Formula (1) according to the embodiment can be a material of an organic luminescent layer in an organic light-emitting diode. A preferred example is the compound of Chemical Formula (1), DiBizAn, where A is the General Formula (2), B is the General Formula (5), and R₁ to R₁₇ and R₂₃ to R₃₁ are independent hydrogen atoms.

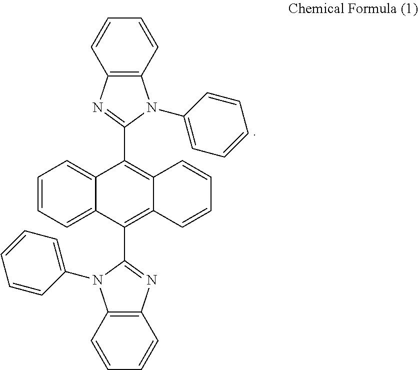

Alternatively, another preferred example is the compound of Chemical Formula (2), monoBizAn, where A is the General Formula (3), B is the General Formula (5), R₁ to R₈ and R₁₈ to R₃₁ are independent hydrogen atoms.

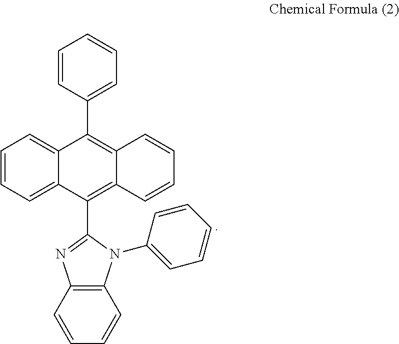

To illustrate the synthesis of Chemical Formula (1) to Chemical Formula (4), there are several examples shown below.

Synthesis of Compound 1 (DiBizAn, Chemical Formula (1))

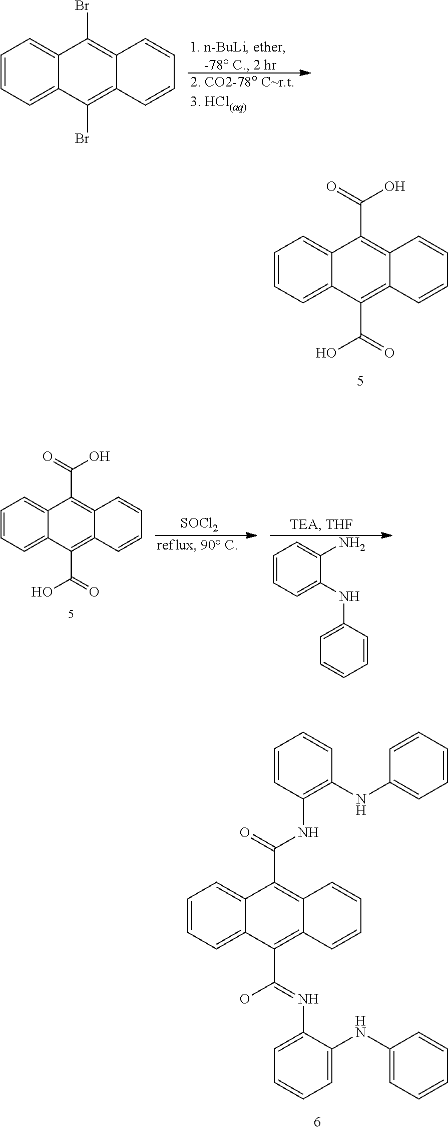

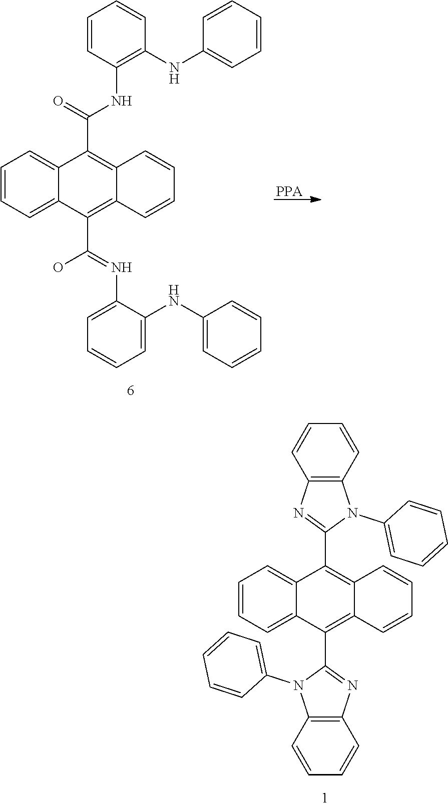 [SMILES: O=C(Nc1ccccc1Nc1ccccc1)c1c2ccccc2c(/C(O)=N/c2ccccc2Nc2ccccc2)c2ccccc12 ; c1ccc(-n2c(-c3c4ccccc4c(-c4nc5ccccc5n4-c4ccccc4)c4ccccc34)nc3ccccc32)cc1]

### Example 1: Synthesis of Compound 5 (anthracene-9,10-dicarboxylic Acid)

9,10-dibromoanthracene (10 g, 2.98 mmol) and a stir were provided in a 500 ml two-neck bottle. After installing a dropping funnel and a three-way valve, and purged with argon for three times, dehydrated ether (100 ml) was added into the bottle from the dropping funnel. The mixture was stirred and cooled to −78° C. under iced bath (dry ice and acetone), and then n-BuLi (53 ml, 8.48 mmol) was added. After removing the iced bath and returned to room temperature, the mixture was stirred for 2 hours and then returned to the iced bath. After removing the dropping funnel and rapidly adding exceed dry ice, the mixture was returned to room temperature slowly. 2M HCl (60 ml) was added, and the mixture was stirred to generate yellow powder, which was filtered. The obtained yellow powder was added to 120 ml acetone and heated to reflux for 1 hour. 300 ml n-hexane was added, and the solution was filtered to obtain compound 5 (4.21 g, yield: 63%) as a yellow solid. The foregoing reaction is as shown in the Reaction Formula (1).

The above synthesis can refer to the reference: (Quah, Hong Sheng; Ng, Li Ting; Donnadieu, Bruno; Tan, Geok Kheng; Vittal, Jagadese J. Inorganic Chemistry, 2016, vol. 55, #21 p. 10851-10854).

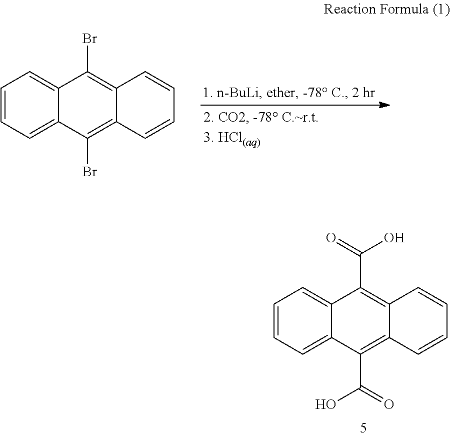 [SMILES: Brc1c2ccccc2c(Br)c2ccccc12 ; O=C(O)c1c2ccccc2c(C(=O)O)c2ccccc12]

### Example 2: Synthesis of Compound 6 (N⁹,N¹⁰-bis(2-(phenylamino)phenyl)anthracene-9,10-dicarboxamide)

Anthracene-9,10-dicarboxylic acid (compound 5, 1.455 g, 5.46 mmol) was provided in a 25 ml round bottom bottle, and thionyl chloride (7 ml, 9.65 mmol) was added. After heating to reflux for 12 hours, the acyl chlorination was finished, and then the thionine chloride was removed. The mixture was vacuumed and dissolved in anhydrous THF (30 ml), and then slowly added into a 100 ml two-neck bottle, which was provided with N-phenyl-1,2-benzenediamine (2.03 g, 11.02 mmol) and dehydrated triethyl amine (3.04 ml, 21.81 mmol). After stirring for 16 hours at room temperature, the product was precipitated, THF was removed, and the solution was extracted twice with ethyl acetate (EA) and 1M HCl. The solution was filtered with suction and rinsed several times with deionized water. The product was placed in a round bottom bottle and heated to reflux with a small amount of EA for 2 hours. The solid was obtained by filtration, and hot washed twice to obtain compound 6 (1.91 g, yield: 58%) as an earthy yellow powder. The foregoing reaction is as shown in the Reaction Formula (2).

Spectral data as follow: ¹H NMR (400 MHz, d₆-DMSO): δ 10.45-10.25 (m, 1H), 8.05-8.02 (m, 4H), 7.93 (d, J=7.6 Hz, 2H), 7.49-7.46 (m, 4H), 7.39-7.21 (m, 12H), 6.87-6.80 (m, 6H); ¹³C NMR (100 Mhz, d₆-DMSO): δ 167.09, 144.72, 136.15, 133.85, 130.29, 129.23, 126.81, 126.69, 126.40, 125.80, 125.32, 122.79, 119.23, 115.65.

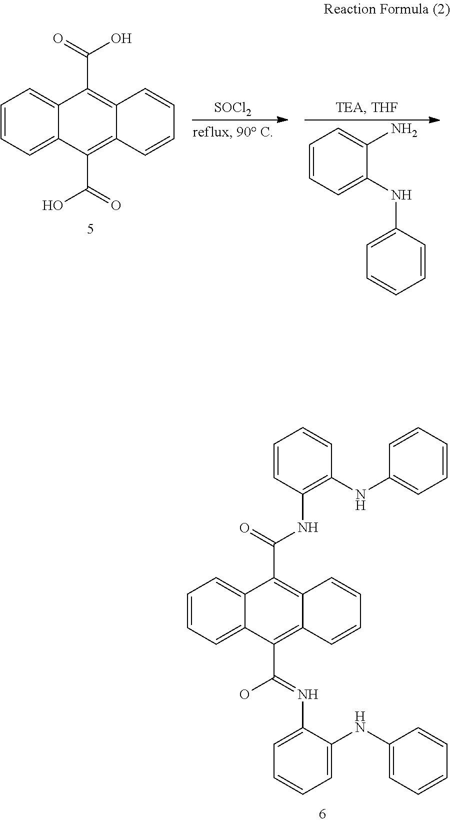

### Example 3: Synthesis of Chemical Formula (1) (DiBizAn)—Compound 1 (9,10-bis(1-phenyl-1H-benzo[d]imidazol-2-yl)anthracene)

Compound 6 (1.89 g, 3.16 mmol) was provided in a round bottom bottle, and polyphosphoric acid (PPA, 23 g) was added. After stirring and heating to 100° C. for 20 hours, the viscous liquid inside the round bottom bottle was dropped into 1M KOH aqueous solution (200 ml) stepwise to generate yellow-brown suspension. After suction filtration, the product was heated to reflux with toluene for 12 hours to maintain the product in the same configuration, and then purified by column chromatography (eluent liquid: dichloromethane) to obtain compound 1 (DiBizAn, Chemical Formula (1), 1.28 g, yield: 71%) as a light yellow solid. The foregoing reaction is as shown in the Reaction Formula (3).

Spectral data as follow: ¹H NMR (400 MHz, CDCl₃) δ 7.98 (d, J=7.6 Hz, 2H), 7.65-7.62 (m, 4H), 7.47-7.37 (m, 6H), 7.34-7.31 (m, 4H), 7.08 (s, 10H); NMR (100 Mhz, CD₂Cl₂) δ 150.77, 144.07, 136.76, 136.21, 131.39, 129.87, 129.67, 128.89, 128.06, 127.31, 126.54, 126.51, 126.38, 124.24, 123.57, 120.60, 111.35, 111.22. HRMS (ESI) m/z calcd for C₄₀H₂₆N₄ 563.22, obsd. 563.2286. Anal Calcd for C₄₀H₂₆N₄: C, 85.38; H, 4.66; N, 9.96. Found: C, 85.09; H, 4.36; N, 10.08.

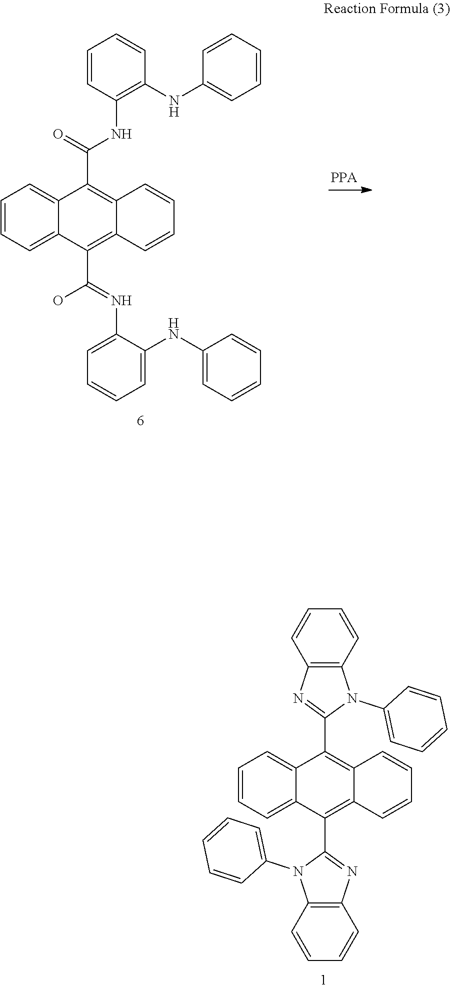 [SMILES: O=C(Nc1ccccc1Nc1ccccc1)c1c2ccccc2c(/C(O)=N/c2ccccc2Nc2ccccc2)c2ccccc12 ; c1ccc(-n2c(-c3c4ccccc4c(-c4nc5ccccc5n4-c4ccccc4)c4ccccc34)nc3ccccc32)cc1]

Synthesis of Compound 2 (monoBizAn, Chemical Formula (2))

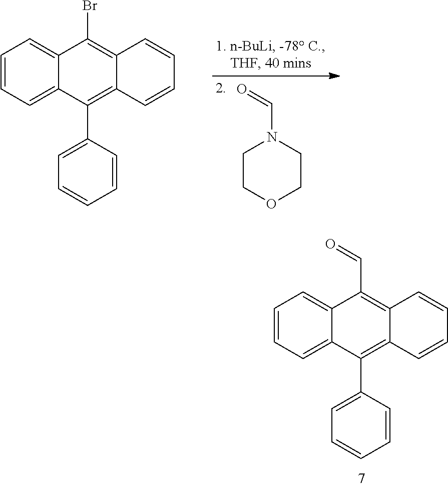 [SMILES: Brc1c2ccccc2c(-c2ccccc2)c2ccccc12 ; O=CN1CCOCC1 ; O=Cc1c2ccccc2c(-c2ccccc2)c2ccccc12]

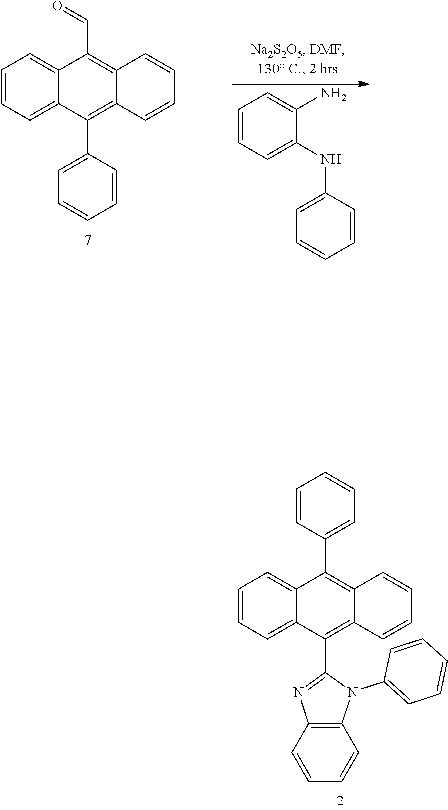 [SMILES: O=Cc1c2ccccc2c(-c2ccccc2)c2ccccc12 ; Nc1ccccc1Nc1ccccc1 ; c1ccc(-c2c3ccccc3c(-c3nc4ccccc4n3-c3ccccc3)c3ccccc23)cc1]

### Example 1: Synthesis of Compound 7 (10-phenylanthracene-9-carbaldehyde)

10-phenyl-9-bromoanthracene (3.00 g, 9.00 mmol) was provided in a two-neck bottle. After installing a dropping funnel and a three-way valve, and purged with argon for three times, anhydrous THF (30 ml) was added into the bottle. The mixture was stirred and cooled to −78° C., and then n-BuLi (6.50 ml, 10.40 mmol) was added slowly. After stirring for 40 minutes, N-formylmorpholine (1.05 ml, 10.44 mmol) was added, and the mixture was stirred for 4 hours at −78° C. After returned to room temperature and stirring for 10 hours, 1M HCl aqueous solution (2 ml) was added, and THF was removed. The solution was extracted with DCM, deionized water and saturated NaCl solution, respectively, and the organic layer was dried with anhydrous MgSO₄. The solution was purified through by column chromatography with n-hexane/DCM=2/1 as eluent to obtain compound 7 (1.85 g, yield: 73%) as a golden solid. The foregoing reaction is as shown in the Reaction Formula (4).

Spectral data as follow: ¹H NMR (400 MHz, CDCl₃) δ 11.53 (s, 1H), 8.95 (d, J=9.2 Hz, 2H), 7.69 (dt, J1=8.8 Hz, J2=1.2 Hz), 7.65-7.57 (m, 5H), 7.41-7.35 (m, 4H); ¹³C NMR (100 Mhz, CDCl₃) δ 193.30, 145.37, 138.08, 131.56, 130.50, 129.79, 128.52, 128.38, 127.96, 127.93, 125.42, 124.94, 123.37. HRMS (FAB) calcd for C₂₁H₁₄O 282.1045, obsd. 282.1043.

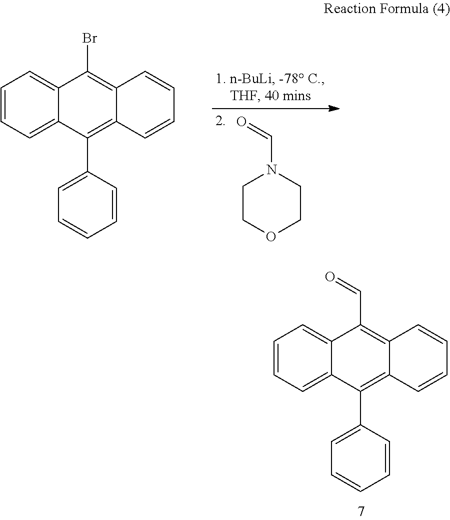 [SMILES: Brc1c2ccccc2c(-c2ccccc2)c2ccccc12 ; O=CN1CCOCC1 ; O=Cc1c2ccccc2c(-c2ccccc2)c2ccccc12]

### Example 5: Synthesis of Chemical Formula (2) (monoBizAn)—Compound 2 (1-phenyl-2-(10-phenylanthracen-9-yl)-1H-benzo[d]imidazole)

Compound 7 (1.45 g, 5.15 mmol), N-phenyl-1,2-benzenediamine (0.95 g, 5.14 mmol), and Na₂S₂O₅ (1.27 g, 6.69 mmol) were provided in a 25 ml round bottom bottle. After purged with argon for three times, dehydrated N,N-dimethylformamide (DMF, 10 ml) were placed in a microwave reactor (reaction conditions: heating up to 130° C. within 1 minute, stirring for 2 hours with 150 W at 130° C.). After the solution was cooled, the solution was added into deionized water to get orange precipitation, which was suction filtered and extracted with trichloromethane, deionized water and saturated NaCl solution, respectively, and the organic layer was dried with anhydrous MgSO₄. The solution was purified through by column chromatography with n-hexane/DCM=1/2 as eluent to obtain yellow solid. After sublimation, the organic product was stirred and rinsed with a small amount of acetone, filtered and sublimed once to obtain a compound 2 (monoBizAn, Chemical Formula (2), 1.24 g, yield: 54%) as a yellow powder. The foregoing reaction is as shown in the Reaction Formula (5).

Spectral data as follow: ¹H NMR (400 MHz, CD₂Cl₂) δ 7.99 (d, J=8 Hz, 1H), 7.69 (d, J=8.6 Hz, 2H), 7.64 (d, J=8.7 Hz, 2H), 7.61-7.56 (m, 3H), 7.49-7.38 (m, 8H), 7.34-7.30 (m, 2H), 7.21-7.19 (m, 2H), 7.16-7.14 (m, 3H); ¹³C NMR (100 Mhz, CD₂Cl₂) δ 151.38, 144.14, 140.55, 138.74, 136.64, 136.47, 131.71, 131.63, 131.33, 129.98, 129.60, 128.85, 128.73, 128.45, 128.15, 127.65, 126.86, 126.48, 126.05, 125.64, 125.14, 123.88, 123.28, 120.47, 111.11. HRMS (ESI) m/z calcd for C₃₃H₂₂N₂ 447.1861 (M⁺) obsd. 447.1899. Anal. Calcd for C₃₃H₂₂N₂: C, 88.76; H, 4.97; N, 6.27; Found: C, 88.26; H, 4.98; N, 6.41.

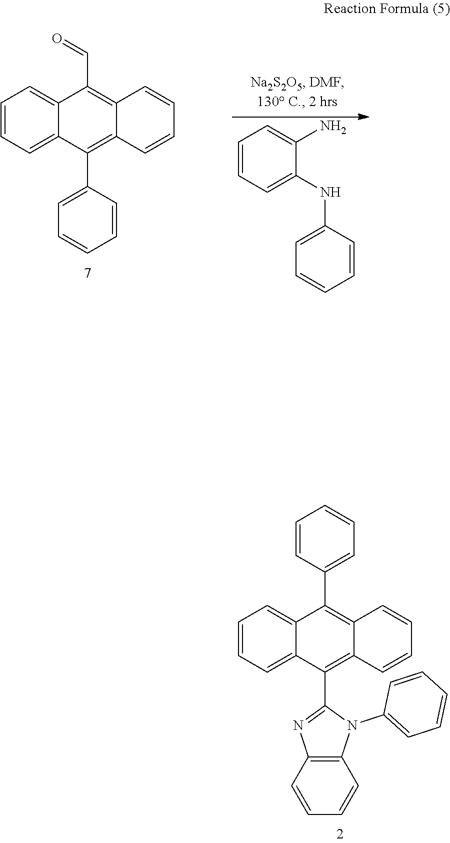 [SMILES: O=Cc1c2ccccc2c(-c2ccccc2)c2ccccc12 ; Nc1ccccc1Nc1ccccc1 ; c1ccc(-c2c3ccccc3c(-c3nc4ccccc4n3-c3ccccc3)c3ccccc23)cc1]

### Example 6: Synthesis of Chemical Formula (3) (PhBorAn)—Compound 3 (4,4,5,5-tetramethyl-2-(10-phenylanthracen-9-yl)-1,3,2-dioxaborolane)

10-phenyl-9-bromoanthracene (1.51 g, 4.54 mmol) was provided in a 50 ml two-neck bottle. After installing a dropping funnel and a three-way valve, and purged with argon for three times, anhydrous THF (22.5 ml) was added into the bottle. The mixture was stayed at −78° C., and then n-BuLi (3.3 ml, 5.28 mmol) was added slowly. After stirring for 1 hour at −78° C. 2-isopropoxy-4,4,5,5-tetramethyl-1,3,2-dioxaborolane (1 ml, 4.9 mmol) was added, and the mixture was returned to room temperature and stirred for 4 hours. 2M HCl (2 ml) was added to quench reaction, and THF was removed. The solution was extracted with trichloromethane, deionized water and saturated NaCl solution, respectively, and the organic layer was dried with anhydrous MgSO₄. The solution was purified through by column chromatography with n-hexane/DCM=2-1 (gradually changed) as eluent, and re-crystalized with DCM and n-hexane to obtain compound 3 (PhBorAn, Chemical Formula (3), 1.07 g, yield: 62%) as a white solid. The foregoing reaction is as shown in the Reaction Formula (6).

Spectral data as follow: ¹H NMR (400 MHz, CDCl₃) δ 8.42 (d, J=8.8 Hz, 2H), 7.62 (d, J=8.8 Hz, 2H), 7.57-7.51 (m, 3H) 7.48-7.43 (m, 2H), 7.39-7.37 (m, 2H), 7.32-7.28 (m, 2H); ¹³C NMR (100 Mhz, CDCl₃) δ 139.56, 139.12, 135.36, 131.07, 130.00, 128.37, 128.28, 127.38, 125.40, 124.80, 84.47. HRMS (FAB) m/z calcd for C₂₆H₂₅BO₂ 380.1948 obsd. 380.1946. Anal. Calcd for C₂₆H₂₅BO₂: C, 82.12; H, 6.63. Found: C, 81.63; H, 6.08.

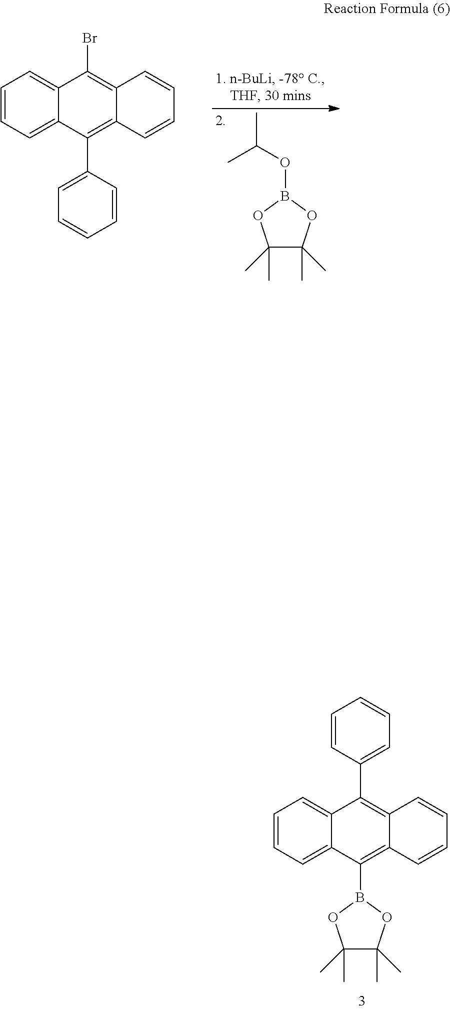 [SMILES: Brc1c2ccccc2c(-c2ccccc2)c2ccccc12 ; CC(C)OB1OC(C)(C)C(C)(C)O1 ; CC1(C)OB(c2c3ccccc3c(-c3ccccc3)c3ccccc23)OC1(C)C]

### Example 7: Synthesis of Chemical Formula (4) (NpBorAn)—Compound 4 (4,4,5,5-tetramethyl-2-(10-(naphthalen-2-yl)anthracen-9-yl)-1,3,2-dioxaborolane)

10-(2-naphtyl)-9-bromoanthracene (3.00 g, 7.83 mmol) was provided in a 50 ml two-neck bottle. After installing a dropping funnel and a three-way valve, and purged with argon for three times, anhydrous THF (26 ml) was added into the bottle. The mixture was stayed at −78° C., and then n-BuLi (5.9 ml, 9.39 mmol) was added slowly. After stirring for 40 minutes at −78° C., 2-isopropoxy-4,4,5,5-tetramethyl-1,3,2-dioxaborolane (1.9 ml, 9.39 mmol) was added, and the mixture was returned to room temperature and stirred for 4 hours. 2M HCl (2 ml) was added to quench reaction, and THF was removed. The solution was extracted with trichloromethane, deionized water and saturated NaCl solution, respectively, and the organic layer was dried with anhydrous MgSO₄. The solution was purified through by silica column chromatography with n-hexane/DCM=3/1 as eluent, and re-crystalized with DCM and n-hexane to obtain compound 4 (NpBorAn, Chemical Formula (4), 2.11 g, yield: 63%) as a light yellow solid. The foregoing reaction is as shown in the Reaction Formula (7).

Spectral data as follow: ¹H NMR (400 MHz, CD₂Cl₂) δ 8.45 (d, J=8.8 Hz, 2H), 8.08-8.02 (m, 2H), 7.93-7.91 (m, 2H), 7.66 (d, J=8.8 Hz, 2H), 7.61-7.58 (m, 2H), 7.53-7.47 (m, 3H), 7.33-7.29 (m, 2H); ¹³C NMR (100 Mhz, CD₂Cl₂) δ 139.67, 136.94, 135.65, 133.79, 133.19, 130.34, 129.70, 128.94, 128.40, 128.26, 128.21, 127.73, 126.83, 126.61, 125.74, 125.37, 84.98. Anal. Calcd for C₃₀H₂₇BO₂: C, 83.73; H, 6.32; B, 2.51; O, 7.44. Found: C, 83.70; H, 6.41.

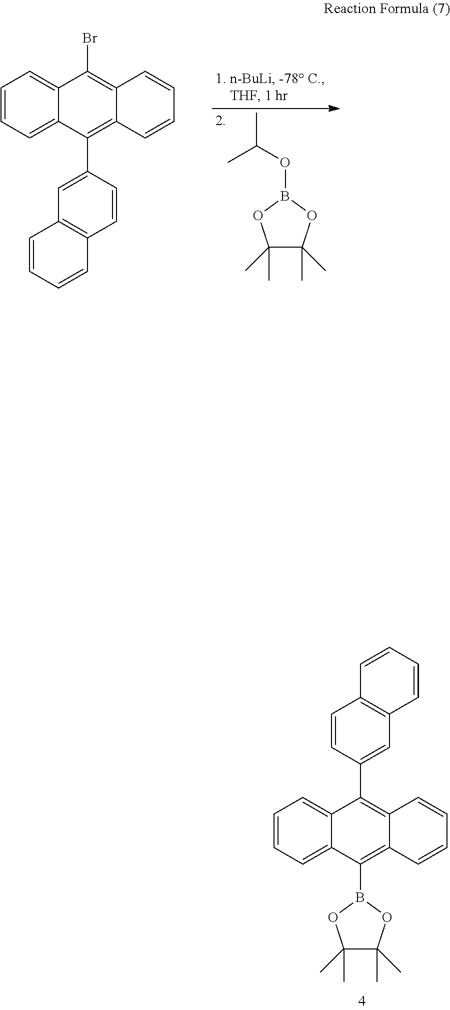 [SMILES: Brc1c2ccccc2c(-c2ccc3ccccc3c2)c2ccccc12 ; CC(C)OB1OC(C)(C)C(C)(C)O1 ; CC1(C)OB(c2c3ccccc3c(-c3ccc4ccccc4c3)c3ccccc23)OC1(C)C]

Evaluation Methods for Using an Organic Electroluminescent Material Containing Anthracene Group as the Material of an Organic Light-Emitting Diode

The material of an organic light-emitting diode includes the compound which is one of the above-mentioned Example 3 and Example 5 to Example 7 (compounds 1 to 4, i.e., Chemical Formulas (1) to (4)). The evaluation method for the material of an organic light-emitting diode is to discuss its thermal, photophysical and electrochemical properties, such as melting point (Tₘ), thermal decomposition temperature (T_{d}), glass transition temperature (T_{g}), maximum absorption wavelength (λₘₐₓ^{abs}) maximum emission peak wavelength (λₘₐₓ^{FL}) of normal temperature fluorescence, maximum emission peak wavelength of low temperature fluorescence (λₘₐₓ^{LTFL}), absorption wavelength start value (λₒₙₛₑₜ^{abs}), fluorescence light quantum yield (PLQY), oxidation potential (E_{DPV}^{ox}), reduction potential (E_{DPV}^{re}), highest occupied molecular orbital energy level (E_{HOMO}), lowest unoccupied molecular orbital energy level (E_{LUMO}), energy gap (E_{g}^{sol}), and triplet annihilation upconversion (TTA-UC).

The maximum absorption wavelength (λₘₐₓ^{abs}), the maximum emission peak wavelength of normal temperature fluorescence (λₘₐₓ^{FL}), and the absorption wavelength start value (λₒₙₛₑₜ^{abs}) are measured by using tetrahydrofuran (10⁻⁵ M) as the solvent, and the maximum emission peak wavelength of low-temperature fluorescence (λₘₐₓ^{LTFL}) is measured by using 2-methyltetrahydrofuran (10⁻⁵ M) as the solvent. They are measured at the temperature of 77K. The fluorescence light quantum yield (PLQY) is measured with a fluorescence spectrometer.

The surface configuration stability of the film plays an important role in the process of fabricating components. The melting point and the glass transition temperature are measured by differential scanning calorimeter (DSC), and the thermal decomposition temperature is measured by thermogravimetric analyzer (TGA), which is considered to be the basis of the stability for the fabrication and performance of unit.

The electrochemical properties of the compound (E_{HOMO}, E_{LUMO}) were obtained by scanning the oxidation potential and reduction potential (E_{DPV}^{ox}, E_{DPV}^{re}) thereof with utilizing cyclic voltammetry (CV) and differential-pulse voltammetry (DPV). In this experimental example, ferrocene was used as a standard. The samples were dissolved in dichloromethane solution, wherein a platinum electrode is used as a working electrode, a platinum wire electrode is used as an auxiliary electrode, and a silver/silver chloride is used as a reference electrode (a three-electrode system) for measuring oxidation potential. In addition, in anhydrous dimethylformamide solvent, a glassy carbon electrode is used as a working electrode for measuring reduction potential. The energy gap (E_{g}^{sol}) is the difference between the highest occupied molecular orbital energy level (E_{HOMO}) and the lowest unoccupied molecular orbital energy level (E_{LUMO}). Understanding E_{HOMO} and E_{LUMO} of a compound can help find a charge injection or transport material that matches the energy gap, thereby making the component more efficient.

The triplet-triplet annihilation upconversion (TTA-UC) utilizes 2,3,7,8,12,13,17,18-Octaethyl-21H,23H-porphine palladium(II) (PdOEP, 10⁻⁵ M) as a sensitizer. The compounds of the present disclosure were subjected as an acceptor (acceptor concentration is 10⁻⁴ M) with using xylene as a solvent, and a green fluorescent pen (λₑₓ=532±10 nm) is used as an excitation light source for testing. After the solution is deoxidized with Ar_{(g)}, the green light is provided to excite the sensitizer to generate a singlet excited state. Since the sensitizer contains palladium (Pd), the intersystem crossing the triplet excited state can be quickly performed. Then, the triplet excited state energy is transferred to the compound in triplet state to be tested, and finally the exciton is converted to the higher energy singlet excited state by TTA-UC so as to emit fluorescence. Therefore, it is possible to excite with the green light having longer wavelength to generate the blue light with shorter wavelength.

The thermal properties of compounds 1 to 4 (Chemical Formula (1) to Chemical Formula (4)) are listed in the following Table 1.

TABLE 1

| Compound | M.W. | Tₘ (° C.) | T_{d}^{a} (° C.) | T_{g} (° C.) |
|---|---|---|---|---|
| Chemical Formula (1) | 562.68 | >350 | 364 | N.D^{b} |
| Chemical Formula (2) | 446.55 | 234 | 310 | N.D^{b} |
| Chemical Formula (3) | 380.29 | 166 | 225 | N.D^{b} |
| Chemical Formula (4) | 430.35 | 209 | 293 | N.D^{b} |

Wherein, ^{a} indicates that the thermal decomposition temperature was accompanied by a weight loss of 5%, and ^{b} indicates that the data were not measured.

The optical properties of compounds 1 to 4 (Chemical Formula (1) to Chemical Formula (4)) are listed in the following Table 2.

TABLE 2

| | λₘₐₓ^{abs} | λₘₐₓ^{FL} | λₘₐₓ^{LTFL} | λₒₙₛₑₜ^{Abs} | |
|---|---|---|---|---|---|
| Compound | (nm) | (nm) | (nm) | (nm) | PLQY^{a} |
| Chemical Formula (1) | 376 | 448 | 414 | 417 | 0.89 |
| Chemical Formula (2) | 375 | 444 | 406 | 412 | 0.88 |
| Chemical Formula (3) | 371 | 429 | 424 | 405 | 0.45 |
| Chemical Formula (4) | 372 | 430 | 424 | 408 | 0.25 |
| DPA | 373 | 409 | 403 | 406 | 0.95 |

Wherein, ^{a} indicates that the data were calculated with the formula: PLQY = Q_{R} × (I/I_{R}) × (OD_{R}/OD) × (n/n_{R}²), DPA was measured in ethanol, and compounds 1 to 4 are measured in THF.

The electrochemical properties of compounds 1 to 4 (Chemical Formula (1) to Chemical Formula (4)) are listed in the following Table 3.

TABLE 3

| | | | E_{HOMO}/E_{LUMO} | E_{g}^{sol} | ^{a}TTA-UC |
|---|---|---|---|---|---|
| Compound | E_{DPV}^{ox} | E_{DPV}^{re} | (eV) | (eV) | test |
| Chemical Formula (1) | 1.01 | −1.92 | −6.01/−3.03 | 2.98 | Blue |
| Chemical Formula (2) | 0.87 | −2.10 | −5.84/−2.87 | 2.97 | Blue |
| Chemical Formula (3) | 0.85 | −2.23 | −5.82/−2.75 | 3.07 | Blue |
| Chemical Formula (4) | 0.79 | −2.18 | −5.75/−2.79 | 2.96 | Blue |
| DPA | 0.73 | −2.11 | −5.68/−2.86 | 2.82 | — |

Wherein, ^{a} indicates that the compound was subjected to TTA-UC test, and Blue indicates that the green fluorescent light was converted to the blue fluorescent light.

According to Table 1, the thermal decomposition temperatures of the Chemical Formulas (1) to (4) are all above 220° C. It is presumed that because the structures thereof all contain a polyphenyl ring structure and have a rigid structure, so the compounds will not have thermal decomposition in high temperature during the heating process. According to Table 3, the compounds of Chemical Formula (1) to Chemical Formula (4) can carry out TTA-UC, so the triplet excitons thereof can be converted back to the singlet state to increase the fluorescence light quantum yield. Based on the above measurement results, the compounds of Chemical Formula (1) to Chemical Formula (4) have good thermal stability and high fluorescence light quantum yield, and have the potential to be a fluorescent material for organic light-emitting diodes.

The Efficiency of Chemical Formula (3) which was Used in an Organic Light-Emitting Diode

The unit structure is ITO/PEDOT:PSS/PVK and Chemical Formula (3)/Mg (2 nm)/Ag (100 nm). The fluorescent material of the organic luminescent layer is made by mixing Chemical Formula (3) and PVK (in different mixing ratios). The material of the first electrode layer of the organic light-emitting diode is ITO. The material of the second electrode layer is aluminum with the thickness of 100 nm. The material of the hole injection layer is PEDOT:PSS. The organic luminescent layer contains 10 mg PVK. The material of the electron transport layer is Mg with the thickness of 2 nm. The organic luminescent layer is formed by spin coating, and the other layers are formed by vapor deposition to manufacture the organic light-emitting diodes of the embodiment, and the driving voltage, the turn-on voltage, the maximum luminance (Luminance, cd/m²), the maximum current efficiency CE (cd/A), and the maximum power efficiency PE (lm/W) of the organic light-emitting diode are measured. The results are shown in Table 4.

TABLE 4

| ^{a} Chemical | ^{b} Turn-on | | | |
|---|---|---|---|---|
| Formula | voltage | L | CE | PE |
| (3) | (V) | (cd/m²) | (cd/A) | (lm/W) |
| 20% | 11.5 | 213 | 0.31@14.0 V | 0.073@13.5 V |
| 30% | 11.0 | 226 | 0.35@14.0 V | 0.081@13.0 V |
| 40% | 9.5 | 281 | 0.39@12.0 V | 0.110@10.5 V |

Wherein, ^{a} indicates the doping concentration of Chemical Formula (3) (weight percentage), and ^{b} indicates the turn-on voltage of the unit under 1 cd/m².

The organic light-emitting diodes, which utilize Chemical Formula (3) (in different ratios) as the fluorescent material, shown in Table 4 have the fine maximum current efficiency and maximum power efficiency. In particular, the organic light-emitting diode containing 40% of Chemical Formula (3) has better efficiencies than other ratios of doped Chemical Formula (3). Accordingly, the fluorescent materials of the present disclosure can be used to make the organic light-emitting diodes with good efficiency.

Efficiency of Chemical Formula (2) and Chemical Formula (4) which were Used in Organic Light-Emitting Diodes

The unit structure is ITO/NPB (60 nm)/EML (40 nm)/BPhen (30 nm)/LiF (1 nm)/Al (100 nm). The fluorescent material of the organic luminescent layer EML is made by Chemical Formula (2) or Chemical Formula (4). Herein, AND is used as the reference material. The material of the first electrode layer of the organic light-emitting diode is ITO. The material of the second electrode layer is aluminum with the thickness of 100 nm. The material of the hole transport layer is NPB. The thickness of the organic luminescent layer is 40 nm. The material of the electron transport layer is BPhen with the thickness of 30 nm. The material of the electron injection layer is LiF with the thickness of 1 nm. The above-mentioned layers are made by vapor deposition to form the organic light-emitting diodes of the embodiment, and the efficiency items and external quantum efficiency (EQE, %) of the units are evaluated. The results are shown in Table 5.

TABLE 5

| | ^{a}Turn-on | | | |
|---|---|---|---|---|
| | voltage | CE | PE | EQE |
| EML | (V) | (cd/A) | (lm/W) | (%) |
| Chemical Formula (2) | 3.0 | 1.05 @ 5.0 V | 0.74 @ 4.0 V | 0.63@ 6.0 V |
| Chemical Formula (4) | 3.5 | 0.53 @ 5.5 V | 0.44 @ 3.5 V | 0.51 @ 6.5 V |
| ADN | 5.0 | 1.65 @ 8.5 V | 0.63 @ 8.5 V | 1.60 @ 8.5 V |

Wherein, ^{a} indicates the turn-on voltage of the unit under 1 cd/m³.

The organic light-emitting diodes, which utilize Chemical Formula (2) and Chemical Formula (4) as the fluorescent material, shown in Table 5 have lower turn-on voltages than the reference material, and have the fine maximum current efficiency, maximum power efficiency, and maximum external quantum efficiency. Accordingly, the fluorescent materials of the present disclosure can be used to make the organic light-emitting diodes with good efficiency.

Evaluation Results of Chemical Formula (2) and Chemical Formula (4) Used for Delayed Fluorescence of Fluorescent Material

The unit structure is ITO/NPB (60 nm)/EML (40 nm)/BPhen (30 nm)/LiF (1 nm)/Al (100 nm). The fluorescent material of the organic luminescent layer EML is made by Chemical Formula (2) or Chemical Formula (4). Herein, the material of the first electrode layer of the organic light-emitting diode is ITO. The material of the second electrode layer is aluminum with the thickness of 100 nm. The material of the hole transport layer is NPB. The thickness of the organic luminescent layer is 40 nm. The material of the electron transport layer is BPhen with the thickness of 30 nm. The material of the electron injection layer is LiF with the thickness of 1 nm. The above-mentioned layers are made by vapor deposition to form the organic light-emitting diodes of the embodiment. The units are subjected to the evaluation of transient electroluminescence (TrEL), and compared with the commercial TTA-UC luminescent material AND for evaluating whether the units have delayed fluorescence phenomenon. The results are shown in Table 6.

TABLE 6

| EML | τ₁ (μs) | τ₂ (μs) |
|---|---|---|
| Chemical Formula (2) | 0.85 | 1.88 |
| Chemical Formula (4) | 0.43 | —^{a} |
| ADN | 0.69 | 2.15 |

Wherein, τ₁ (μs) indicates fluorescence emission (single-state exciton), τ₂ (μs) indicates delayed fluorescence emission (two triplet excitons of TTA return to singlet state), and ^{a} indicates it is not measured.

According to the evaluation results of transient electroluminescence as shown in Table 6, the organic light-emitting diodes, which utilize Chemical Formula (2) and Chemical Formula (4) as the fluorescent material, have lower delayed fluorescence than AND. In particular, the organic light-emitting diodes, which utilize Chemical Formula (4) as the fluorescent material, have much lower or unmeasured delayed fluorescence phenomenon than AND. Accordingly, the fluorescent materials of the present disclosure can be used to make the organic light-emitting diodes with less or no delayed fluorescence phenomenon.

As mentioned above, in the electroluminescent materials containing anthracene group and organic light-emitting diodes by using the same according to the present disclosure, it utilizes anthracene as a core structure. Benzimidazole having electron transport function is introduced to synthesize the electroluminescent materials containing anthracene group, which have good fluorescence quantum performance and thermal stability. Thus, the electroluminescent materials containing anthracene group are suitable for manufacturing the organic light-emitting diodes with good fluorescence quantum performance and thermal stability.

Although the disclosure has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the disclosure.

What is claimed is:

1. An organic light-emitting diode, comprising:
a first electrode layer;
a second electrode layer; and
an organic luminescent unit disposed between the first electrode layer and the second electrode layer, wherein the organic luminescent unit has an organic electroluminescent material containing anthracene group as shown in General Formula (1): 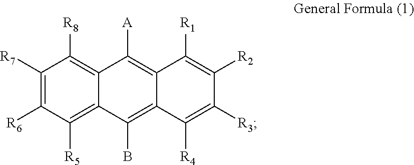
wherein A is selected from the group consisting of General Formula (3) and General Formula (4): 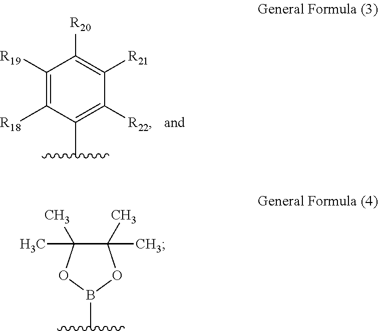
wherein B is selected from the group consisting of General Formula (5), General Formula (6) and General Formula (7): 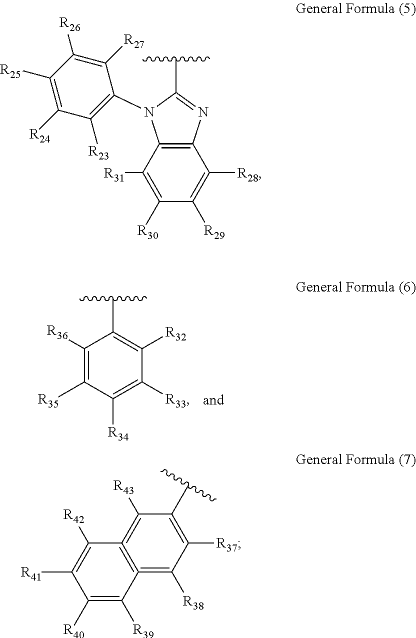
wherein B is General Formula (5) when A is General Formula (3);
wherein B is selected from the group consisting of General Formula (6) and General Formula (7) when A is General Formula (4); and
wherein R₁ to R₈ and R₁₈ to R₄₃ are independently selected from the group consisting of hydrogen atom, fluorine atom, cyano group, alkyl group, cycloalkyl group, alkoxy group, haloalkyl group, thioalkyl group, silyl group and alkenyl group.

2. The organic light-emitting diode according to claim 1, wherein the alkyl group is selected from the group consisting of a substituted or unsubstituted straight-chain C1˜C6 alkyl group, and a substituted or unsubstituted branched-chain C3˜C6 alkyl group, the cycloalkyl group is a substituted or unsubstituted C3˜C6 cycloalkyl group, the alkoxy group is selected from the group consisting of a substituted or unsubstituted straight-chain C1˜C6 alkoxy group, and a substituted or unsubstituted branched-chain C3˜C6 alkoxy group, the haloalkyl group is selected from the group consisting of a substituted or unsubstituted straight-chain C1˜C6 haloalkyl group, and a substituted or unsubstituted branched-chain C3˜C6 haloalkyl group, the thioalkyl group is selected from the group consisting of a substituted or unsubstituted straight-chain C1˜C6 thioalkyl group, and a substituted or unsubstituted branched-chain C3˜C6 thioalkyl group, the silyl group is selected from the group consisting of a substituted or unsubstituted straight-chain C1˜C6 silyl group, and a substituted or unsubstituted branched-chain C3˜C6 silyl group, and the alkenyl group is selected from the group consisting of a substituted or unsubstituted straight-chain C2˜C6 alkenyl group, and a substituted or unsubstituted branched-chain C3˜C6 alkenyl group.

3. The organic light-emitting diode according to claim 1, wherein the organic electroluminescent material containing anthracene group comprises a structure of any of the following Chemical Formulas (2) to (4):

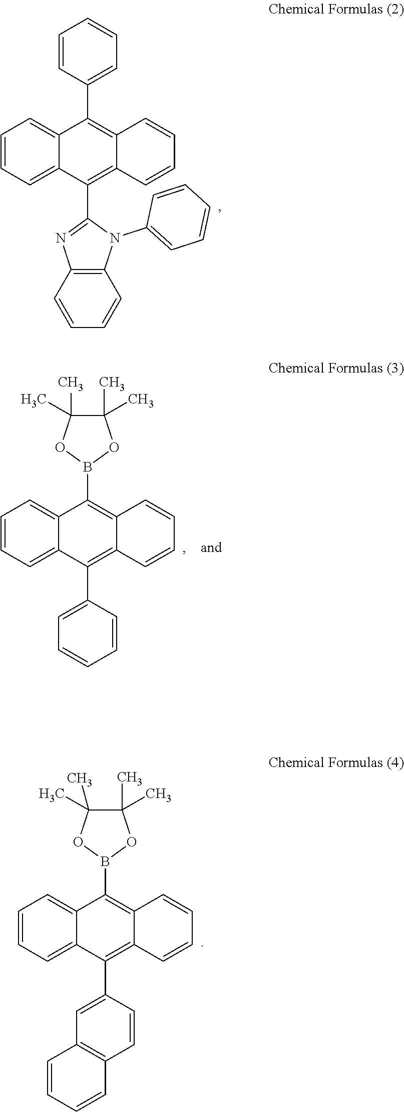 [SMILES: c1ccc(-c2c3ccccc3c(-c3nc4ccccc4n3-c3ccccc3)c3ccccc23)cc1 ; CC1(C)OB(c2c3ccccc3c(-c3ccccc3)c3ccccc23)OC1(C)C ; CC1(C)OB(c2c3ccccc3c(-c3ccc4ccccc4c3)c3ccccc23)OC1(C)C]

4. The organic light-emitting diode of claim 1, wherein the organic luminescent unit comprises an organic luminescent layer.

5. The organic light-emitting diode of claim 4, wherein the organic luminescent unit further comprises a hole transport layer and an electron transport layer, and the organic luminescent layer is disposed between the hole transport layer and the electron transport layer.

6. The organic light-emitting diode of claim 4, wherein the organic luminescent unit further comprises a hole injection layer, a hole transport layer, an electron transport layer and an electron injection layer, and the hole transport layer, the organic luminescent layer and the electron transport layer are sequentially disposed between the hole injection layer and the electron injection layer.

7. The organic light-emitting diode of claim 4, wherein the organic electroluminescent material containing anthracene group is a fluorescent organic electroluminescent material.

8. The organic light-emitting diode of claim 4, wherein the organic luminescent layer comprises polyvinylcarbazole and the organic electroluminescent material containing anthracene group.

9. An organic electroluminescent material containing anthracene group, comprising a structure of the following General Formula (1): 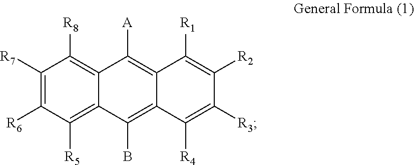
wherein A is General Formula (3): 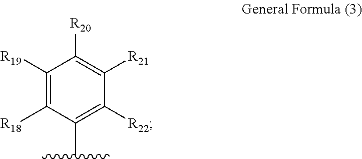
wherein B comprises a structure of General Formula (5): 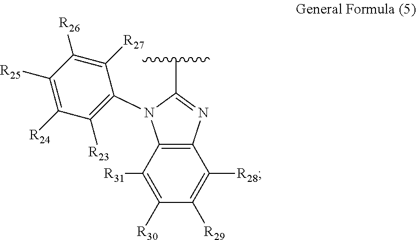
and
wherein R₁ to R₈ and R₁₈ to R₃₁ are independently selected from the group consisting of hydrogen atom, fluorine atom, cyano group, alkyl group, cycloalkyl group, alkoxy group, haloalkyl group, thioalkyl group, silyl group and alkenyl group.

10. The organic electroluminescent material containing anthracene group according to claim 9, wherein the alkyl group is selected from the group consisting of a substituted or unsubstituted straight-chain C1˜C6 alkyl group, and a substituted or unsubstituted branched-chain C3˜C6 alkyl group, the cycloalkyl group is a substituted or unsubstituted C3˜C6 cycloalkyl group, the alkoxy group is selected from the group consisting of a substituted or unsubstituted straight-chain C1˜C6 alkoxy group, and a substituted or unsubstituted branched-chain C3˜C6 alkoxy group, the haloalkyl group is selected from the group consisting of a substituted or unsubstituted straight-chain C1˜C6 haloalkyl group, and a substituted or unsubstituted branched-chain C3˜C6 haloalkyl group, the thioalkyl group is selected from the group consisting of a substituted or unsubstituted straight-chain C1˜C6 thioalkyl group, and a substituted or unsubstituted branched-chain C3˜C6 thioalkyl group, the silyl group is selected from the group consisting of a substituted or unsubstituted straight-chain C1˜C6 silyl group, and a substituted or unsubstituted branched-chain C3˜C6 silyl group, and the alkenyl group is selected from the group consisting of a substituted or unsubstituted straight-chain C2˜C6 alkenyl group, and a substituted or unsubstituted branched-chain C3˜C6 alkenyl group.

11. The organic electroluminescent material containing anthracene group according to claim 9, wherein the organic electroluminescent material containing anthracene group comprises a structure of Chemical Formula (2): 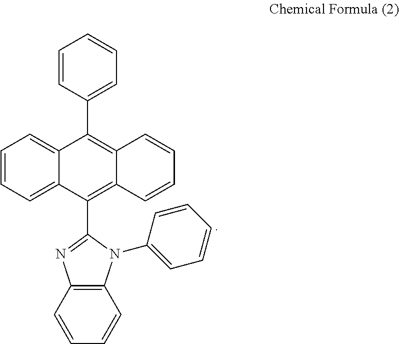 [SMILES: c1ccc(-c2c3ccccc3c(-c3nc4ccccc4n3-c3ccccc3)c3ccccc23)cc1]
